(19) United States
(12) Patent Application Publication
(10) Pub. No.: US 2023/0104692 A1
(43) Pub. Date: Apr. 6, 2023

(54) DMEU ENHANCER

(71) Applicant: LTS LOHMANN THERAPIE-SYSTEME AG, Andemach (DE)

(72) Inventor: Andreas KOCH, Melsbach (DE)

(21) Appl. No.: 17/913,747

(22) PCT Filed: Mar. 22, 2021
(86) PCT No.: PCT/EP2021/057247

§ 371 Date: Sep. 22, 2022

(30) Foreign Application Priority Data
Mar. 23, 2020 (DE) 10 2020 107 937.0

(51) Int. Cl. A61K 9/70; A61K 31/5513; A61K 47/18

(52) U.S. Cl. CPC A61K 9/7069; A61K 31/5513; A61K 47/18

(57) ABSTRACT

The present invention relates to a dosage form for transdermal administration of at least one active pharmaceutical ingredient with a logP≥3, comprising at least one penetration accelerator, wherein the at least one penetration accelerator comprises dimethylethylene urea, the use of such a dosage form as a medicament, and the use of dimethylethylene urea as penetration accelerator to increase the skin penetration of active pharmaceutical ingredients with a logP≥3.

The present invention relates to a dosage form for transdermal administration of at least one active pharmaceutical ingredient with a logP≥3, comprising dimethylethylene urea as penetration accelerator, the use of such a dosage form as medicament, and the use of dimethylethylene urea as penetration accelerator to increase the skin penetration of active pharmaceutical ingredients with a logP≥3.

Dosage forms for transdermally administering at least one active pharmaceutical ingredient have enjoyed widespread use in recent years in the treatment of numerous diseases since they are associated with certain advantages as compared to other administration forms.

On the one hand, for example, the stomach, intestine, and liver are protected due to an avoidance of the gastrointestinal tract. On the other hand, the first-pass effect can be bypassed and the compliance can be increased, since the patient does not have to take tablets at regular intervals. In comparison to oral dosage forms such as tablets, it is also possible to achieve a continuous and controlled release of the active ingredient over a longer period of time, without a risk of overdosing or underdosing.

One disadvantage, however, is that the skin, as an organ of absorption, serves as a protective barrier from its evolutionary development, and thus there are available only a limited number of active ingredients to be applied transdermally which, due to their physicochemical properties, are actually capable of permeating through the skin until they reach the systemic bloodstream.

Permeability is the ability of solids (also porous), especially thin partitions, to be penetrated by certain substances (gases, liquids, dissolved molecules, ions or atoms). In the present case, this means the penetrability of human or animal skin for small molecules, especially for active pharmaceutical ingredients. In technical terms, permeation is the process of one substance crossing or passing through another. The term is often used in conjunction with the passage of cosmetic or pharmaceutical active ingredients into or through the skin.

In order to increase the number of possible active ingredients for transdermal application on the one hand, and in order to ensure that the active ingredients also reach the necessary therapeutic permeation quantity on the other, numerous methods for increasing the permeation quantity are known.

These range from simple measures, such as non-invasive and passive occlusion, to elaborate and active measures, such as iontophoresis or inverse skin pre-perforation using biodegradable microneedle systems containing the active ingredient.

The classic measures include increasing the permeation quantity by means of chemical penetration accelerators.

Penetration accelerators are compounds that are able to penetrate into the uppermost skin layer, the stratum corneum, where they reversibly reduce the resistance of the permeation barrier and thus facilitate the permeation (permeation=complete penetration of all skin layers) of active ingredients of a medicament through the skin or make it possible in the first place.

All potential penetration accelerators should be distinguished in being non-toxic, non-irritating to the skin, and non-allergenic. Above all, they should not produce pharmacological effects on the skin or in the organism. Penetration accelerators should be chemically and physically compatible with the relevant active ingredient of the medicament and other excipients of the dosage form. The ideal penetration accelerator should also be characterised by the fact that it simultaneously acts as a solvent for the relevant active ingredient of the medicament, for example if this, due to its physicochemical properties, does not dissolve in the intended transdermal vehicle.

This is often the case with very lipophilic and thus at the same time poorly water-soluble active ingredients. According to current knowledge, penetration accelerators can be divided into six classes without going into the individual modes of action here.
pure solvents (for example sulfoxide derivatives, dimethylformamide)
alcohols and polyols (for example ethanol, propylene glycol, glycerol)
Azone® derivatives
fatty acids, terpenes, and fatty acid derivatives (for example oleic acid)
urea and urea derivatives
weakly surface-active substances containing polar groups of suitable size (for example anionic surfactants, such as sodium lauryl sulfate).

For a number of very lipophilic and almost water-insoluble active ingredients, especially those with a logP greater than three, it was found that the known penetration accelerators used did not lead to satisfactory permeation rates that would have justified their use in a dosage form for the transdermal application of an active pharmaceutical ingredient. In addition, the successful development of such a dosage form with various active pharmaceutical ingredients failed due to the fact that not enough active ingredient could be dissolved in the respective carrier systems to build up the necessary thermodynamic pressure for the passive diffusion process.

The aim of the present invention was therefore to provide a dosage form for transdermal administration of at least one active pharmaceutical ingredient having a logP≥3, comprising at least one penetration accelerator, with which a satisfactory permeation rate of the at least one active pharmaceutical ingredient into the skin of the patient can be achieved. In addition, the penetration accelerator should be chemically and physically compatible with the active ingredient of the medicament and other excipients of the dosage form. The penetration accelerator should also be characterised in that it acts simultaneously as a solvent for the active pharmaceutical ingredient in question if the latter, for example due to its physicochemical properties, does not dissolve in the intended transdermal vehicle.

This aim has surprisingly been addressed by a dosage form for transdermal administration of at least one active pharmaceutical ingredient comprising at least one active pharmaceutical ingredient with a logP≥3 and at least one penetration accelerator, characterised in that the at least one penetration accelerator comprises dimethylethylene urea.

Hereinafter, the word “comprising” can also mean “consisting of”.

In this text, the terms penetration accelerator, penetration booster, permeation accelerator, permeation booster, penetration intensifier, permeation intensifier and enhancer may be used synonymously.

Dimethylethylene urea (DMEU) means the compound 1,3-dimethyl-2-imidazolidinone of the following formula (I):

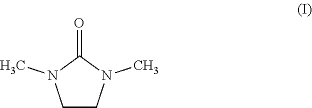

The n-octanol-water partition coefficient K_{ow} (notations such as octanol/water partition coefficient are also common and correct) is a dimensionless partition coefficient known to a person skilled in the art, which indicates the ratio of the concentrations of a chemical in a two-phase system of n-octanol and water and is thus a measure of the hydrophobicity or hydrophilicity of a substance. The logP value is the decadic logarithm of the n-octanol-water partition coefficient K_{ow}. The following is true:

$${K}_{ow} = P = \frac{{c}_{o}^{Si}}{{c}_{w}^{Si}} and log P = log \frac{{c}_{o}^{Si}}{{c}_{w}^{Si}} = log {c}_{o}^{Si} - {c}_{w}^{Si} ,$$

with cₒ^{si}=concentration of a chemical in the octanol-rich phase and

c_{w}^{si}=concentration of a chemical in the water-rich phase.

K_{ow} is greater than one if a substance is more soluble in fat-like solvents such as n-octanol, less than one if it is more soluble in water. Accordingly, logP is positive for lipophilic substances and negative for hydrophilic substances.

The form of the dosage form for transdermally administering at least one active pharmaceutical ingredient is in principle not restricted.

The dosage form according to the invention, however, is preferably characterised in that it comprises a transdermal therapeutic system, a gel, a lotion, an ointment and/or a cream.

A transdermal therapeutic system (also called a transdermal patch) is understood to mean a system to be applied to the skin, preferably a patch, with a defined application area, which can deliver an active pharmaceutical ingredient to a patient's body in a controlled manner, preferably according to time and quantity.

Such systems usually have a cover film (backing layer) as backing that protects the patch and its contents from the external environment and may be printed with information. Towards the skin side, a peel-off film (release liner) is preferably provided, which covers the sticky side of the system. The peel-off film is removed before the system is applied and is often siliconised to facilitate removal.

With regard to the technique of controlled active ingredient delivery from the system, a distinction can be made between matrix systems (matrix patches) and membrane systems (also called reservoir or depot systems or reservoir or depot patches).

In matrix systems, the active ingredient is contained in a matrix which consists of one or more layers and which is applied directly to the skin with the aid of an adhesive layer. Embodiments are also possible in which the matrix is simultaneously the adhesive layer. The diffusion rate of the active ingredient out of the matrix determines the resorption rate. In some embodiments, there may be an additional membrane between the matrix and adhesive layers that controls the flow of active ingredient.

In the membrane systems, a reservoir of the active ingredient lies under a carrier film, wherein the active ingredient is released from the reservoir through a porous membrane into the skin in a controlled manner. In the reservoir, the active ingredient is preferably present as a solution or suspension. Preferably, a carrier material, such as a non-woven fabric, which has been impregnated with this solution and/or suspension, can serve as a reservoir.

The advantages of a transdermal therapeutic system on the patient side are a safe, reliable, exact, and painless dosage of active pharmaceutical ingredients and the easier therapy of children, elderly patients, and patients in need of extra care. Furthermore, transdermal therapeutic systems are ideal for patients with swallowing difficulties and for extended dosing intervals, especially with multi-day patches.

The advantages of a transdermal therapeutic system on the manufacturer's side are the possible formulation of active pharmaceutical ingredients with only low oral bioavailability, a controlled, uniform delivery of active pharmaceutical ingredients without active ingredient peaks, a good possibility to control the medicament dosage by varying the area, no loss of active ingredient by avoiding the first-pass metabolism in the liver, and no degradation of the active ingredient in the gastrointestinal tract.

Gels usually comprise gelled liquids. They are preferably produced with suitable swelling agents (gelling agents). These include, for example, celluloses, starches, carbomers, gelatine, xanthan, bentonite, agar and/or pectin.

A distinction is made here between hydrophilic and lipophilic gels. Gels can be transparent or opaque.

Other possible ingredients include water, propylene glycol, antioxidants, lipids (in lipogels), flavourings, sweeteners and/or preservatives.

Among other things, gels are used for the local or systemic administration of active ingredients and for moist wound treatment.

A lotion is an externally applied liquid aqueous or aqueous-alcoholic preparation containing suspended or emulsified active pharmaceutical ingredients, and possibly excipients.

Lotions are generally more liquid than creams or ointments and are therefore easier to apply to large areas of the skin.

A lotion is an externally applied oil-in-water emulsion or water-in-oil emulsion. It is very light and does not lubricate.

Lotions are used, among other things, for the local or systemic administration of active ingredients and for moist wound treatment.

An ointment, preferably a suspension ointment, is a semi-solid preparation for external use. Ointments preferably consist of a single-phase base in which solid or liquid substances can be dispersed.

A distinction is made between hydrophobic ointments, water-absorbing ointments, and hydrophilic ointments. Ointments can also contain emulsifiers and water.

For example, fatty oils, fats, waxes, petroleum products such as petrolatum and paraffins, triglycerides and/or macrogols (PEG) can be used to produce ointments.

Ointments are used, among other things, for the local or systemic administration of active ingredients and for moist wound treatment.

A cream is a semi-solid preparation, usually for application to the skin.

A cream is preferably a multi-phase preparation consisting of a lipophilic and an aqueous phase and containing at least one active pharmaceutical ingredient. A distinction is made between a hydrophilic cream (oil-in-water) and a lipophilic/hydrophobic cream (water-in-oil).

A cream is used, among other things, for the local or systemic administration of active ingredients and for moist wound treatment.

The dosage form according to the invention in the form of a gel, a lotion, an ointment and/or a cream is preferably characterised in that the at least one penetration accelerator dimethylethylene urea is present in an amount of from 10 to 30 wt. %, preferably from 12 to 25 wt. %, especially preferably from 15 to 18 wt. %, in relation to the total weight.

The dosage form according to the invention is preferably characterised in that the at least one active pharmaceutical ingredient has a logP>3, preferably greater than 3.2 or 3.4 or 3.6 or 3.8 or 4 or 4.2 or 4.4 or 4.6 or 4.8 or 5 or 6 or 7.

The dosage form according to the invention is preferably characterised in that the at least one active pharmaceutical ingredient with a logP≥3 has a water solubility of less than 0.01 mg/ml (at 20° C.).

Preferably, the water solubility of the at least one active pharmaceutical ingredient is less than 0.005 mg/ml, especially preferably less than 0.001 mg/ml (at 20° C.).

Preferably, the at least one active pharmaceutical ingredient has a molecular weight of more than 300 g/mol, preferably of more than 350 g/mol, or of more than 400 g/mol, especially of more than 450 g/mol.

The dosage form according to the invention is preferably characterised in that the at least one active pharmaceutical ingredient is selected from the group consisting of hypnotics, sedatives, antiepileptics, analeptics, psychoneurotropic drugs, neuroleptics, neuro-muscle blockers, antispasmodics, antihistamines, antiallergics, cardiotonics, antiarrhythmics, diuretics, hypotensives, vasopressors, antitussives, expectorants, analgesics, thyroid hormones, sexual hormones, glucocorticoid hormones, antidiabetics, antitumour drugs, antibiotics, chemotherapeutics, narcotics, anti-Parkinson drugs, anti-Alzheimer drugs and/or triptans.

Especially preferably, the at least one active pharmaceutical ingredient is selected from the group consisting of sedatives. Olanzapine, curcumin and felodipine are mentioned here by way of example.

Especially preferably, the at least one active pharmaceutical ingredient is not selected from the group comprising cannabinoids, such as cannabidiol and/or cannbidivarol.

The dosage form according to the invention is preferably characterised in that the dosage form represents a transdermal therapeutic system, wherein the transdermal therapeutic system has a backing and a matrix layer containing the at least one active pharmaceutical ingredient with a logP≥3. The penetration accelerator dimethylethylene urea is preferably contained in the matrix layer.

The backing is preferably impermeable to the at least one active pharmaceutical ingredient.

The type of backing is not limited. The backing can include plastics films or metal foils, but also knitted fabrics or non-wovens.

Most suitable for the backing are layers or films made of plastic, such as polyethylene terephthalate (PET). The advantage of these plastics layers or plastics films is that they are inexpensive to produce and impermeable to almost all active pharmaceutical ingredients.

The transdermal therapeutic system according to the invention preferably has a removable protective layer on the side of the matrix layer that is not the backing.

In principle, the same materials can be used for the removable protective layer as for the backing, provided that they have been equipped with a removable finish by means of a suitable surface treatment, such as siliconisation.

The transdermal therapeutic system according to the invention is preferably characterised in that the at least one penetration accelerator dimethylethylene urea is provided in the matrix layer in an amount of from 10 to 30 wt. %, preferably of from 12 to 25 wt. %, especially preferably of from 15 to 18 wt. %, or from 18 to 25 wt. % in relation to the active-ingredient-containing matrix layer.

It has been shown that the presence of dimethylethylene urea at these concentrations provides the optimal acceleration of permeation of the at least one active pharmaceutical ingredient.

The transdermal therapeutic system according to the invention is preferably characterised in that the ratio of the penetration accelerator dimethylethylene urea to the at least one active pharmaceutical ingredient is 1:1 or >1:1. Especially suitable are ratios of the penetration accelerator dimethylethylene urea to the at least one active pharmaceutical ingredient of 1.5:1.

It has been shown that such dimethylethylene urea/active ingredient ratios are suitable for maximally loading the TTS with active ingredient so that this dissolves advantageously in the self-adhesive polymer matrix.

The transdermal therapeutic system according to the invention is preferably characterised in that the matrix layer comprises at least one polymer selected from the group consisting of polyacrylates and/or polymethacrylates, natural and/or synthetic rubbers, polysiloxanes, styrene-butadiene block copolymers, isobutylene and/or ethylene-vinyl acetate copolymers.

The transdermal therapeutic system according to the invention is preferably characterised in that the matrix layer comprises at least one polymer selected from the group consisting of silicone-based pressure-sensitive adhesives or silicone pressure-sensitive adhesives, especially amine-resistant silicone pressure-sensitive adhesives.

Preferably, the silicone pressure-sensitive adhesives which can be used in accordance with the invention are pressure-sensitive adhesives based on silicone polymers, such as a dimethiconol/trimethylsiloxysilicate crosspolymer and/or a trimethylsilyl-treated dimethiconol/trimethylsiloxysilicate crosspolymer, which preferably contain at least 30 wt. %, especially 35 to 95 wt. %, especially preferably 40 to 90 wt. %, or 40 to 60 wt. %, or 45 to 55 wt. % of silicone polymer(s), in relation to the silicate.

In one embodiment, the silicone pressure-sensitive adhesives that can be used in accordance with the invention are pressure-sensitive adhesives based on silicone polymers, such as dimethiconol/trimethylsiloxysilicate crosspolymers and/or a trimethylsilyl-treated dimethiconol/trimethylsiloxysilicate crosspolymer, preferably containing 40 wt. % of silicone polymers and 60 wt. % of silicate. Such an adhesive can be referred to by the term “medium-tack adhesives”.

In a further embodiment, the silicone pressure-sensitive adhesives that can be used in accordance with the invention are pressure-sensitive adhesives based on silicone polymers, such as dimethiconol/trimethylsiloxysilicate crosspolymers and/or a trimethylsilyl-treated dimethiconol/trimethylsiloxysilicate crosspolymer, preferably containing 45 wt. % of silicone polymers and 55 wt. % of silicate. Such an adhesive can be referred to as a “high-tack adhesive”.

Mixtures of different silicone pressure-sensitive adhesives can also be used, for example a 1:1 (wt.) ratio of a “medium-tack adhesive” and of a “high-tack adhesive” as described above.

All silicone pressure-sensitive adhesives as described above preferably contain n-heptane as solvent.

The silicone pressure-sensitive adhesives described above are preferably present in an amount of from 50 to 80 wt. %, preferably from about 60 to 75 wt. % solids content in the particular solvent, preferably in n-heptane.

Preferably, the silicone pressure-sensitive adhesives have a peel adhesion of about 700 g/cm for the high-tack adhesive and/or 900 g/cm for the medium-tack adhesive as defined above.

Preferably, the silicone pressure-sensitive adhesives have a shear value of about 14 kg/6.3 cm³ for the high-tack adhesive and about 17 kg/6.3 cm³ for the medium-tack adhesives as defined above.

Preferably, the silicone pressure-sensitive adhesives have a viscosity at 0.01 rad/s and 30° C. (P) of about 5×10⁶ P for the high-tack adhesive and about 1×10⁸ P for the medium-tack adhesives as defined above.

Silicone pressure-sensitive adhesives and especially hot-melt silicone pressure-sensitive adhesives suitable for use in the context of the present invention and as described above are known to a person skilled in the art and are commercially available.

Suitable silicone pressure-sensitive adhesives for use in the present invention include, for example, Dow Corning's BIO-PSA 7-4201 and/or BIO-PSA 4301 hot-melt pressure-sensitive adhesives. In this context, BIO-PSA 7-4201 is a “medium-tack adhesive” and BIO-PSA 7-4301 is a “high-tack adhesive” as defined above.

BIO-PSA 7-4301 is especially preferred, this being a “high-tack adhesive” as defined above.

These polymers are characterised by good compatibility with active pharmaceutical ingredients.

Preferably, these polymers are self-adhesive under application of pressure. This has the advantage that no additional adhesive layer needs to be applied to the matrix to fix the transdermal therapeutic system to the patient's skin.

If a non-self-adhesive polymer is used, the transdermal therapeutic system is preferably fixed to the patient's skin by another adhesive layer.

The transdermal therapeutic system according to the invention is preferably characterised in that the at least one polymer is present in the matrix layer in an amount of from 40 to 98 wt. %, preferably from 50 to 80 wt. %, especially preferably from 60 to 75 wt. %, in relation to the active-ingredient-containing matrix layer, wherein the matrix layer preferably is not to be understood as a single-layer matrix, i.e. the TTS according to the invention is not a multi-layer system.

The dosage form according to the invention is preferably a transdermal therapeutic system which is formed as membrane systems, wherein the at least one active pharmaceutical ingredient is present in the matrix layer in a reservoir, from which the at least one active pharmaceutical ingredient can be dispensed in a controlled manner through a porous control membrane covering the reservoir.

The term ‘matrix layer’ also includes the term ‘reservoir’.

This transdermal therapeutic system is preferably characterised in that the control membrane comprises a polymer film, wherein the polymer forming the basis of the polymer film is selected from polyethylene, polypropylene, polyurethane, silicone and/or copolymers of ethylene and vinyl acetate.

The transdermal therapeutic system according to the invention is preferably characterised in that the matrix layer comprises further excipients, selected from the group consisting of plasticisers, crystallisation inhibitors, stabilisers, antioxidants and/or neutralisers.

Each of these excipients may be present in the matrix layer in an amount of from 0.1 to 10 wt. %, in relation to the weight of the matrix layer.

The transdermal therapeutic system according to the invention is preferably characterised in that the at least one active pharmaceutical ingredient is present in the matrix layer in an amount of from 0.1 to 50 wt. %, in relation to the weight of the matrix layer.

The transdermal therapeutic system according to the invention is preferably characterised in that the transdermal therapeutic system has a loading with the at least one active pharmaceutical ingredient of greater than 6 mg/cm².

The transdermal therapeutic system according to the invention is preferably characterised in that the transdermal therapeutic system has a loading with the at least one active pharmaceutical ingredient of from 6 mg/cm² to 8.5 mg/cm², or from 7.5 to 8.5 mg/cm².

Here, the area preferably refers to the area of the matrix layer or the reservoir.

The penetration accelerator dimethylethylene urea can be used alone or in combination with other penetration accelerators. Other suitable penetration accelerators include fatty acids and/or fatty acid esters, such as pentanoic acid, hexanoic acid, octanoic acid, nonanoic acid, decanoic acid, lauric acid, myristic acid, palmitic acid, stearic acid, arachidic acid, behenic acid, lignoceric acid, isoverlinic acid, neoheptonic acid, neonanoic acid, isostearic acid, oleic acid, palmitoleic acid, linolenic acid, vaccenic acid, petroselinic acid, elaidic acid, oleic acid, arachidonic acid, gadoleic acid, erucic acid, ethyl acetate, methyl propylate, butyl acetate, methyl valerate, diethyl sebacitate, methyl laurate, ethyl oleate, isopropyl decanoate, isopropyl myristate (isopropyl ester of myristic acid), isopropyl palmitate and/or isopropyl oleate.

Furthermore, the dosage form according to the invention is preferably characterised in that no penetration accelerators from the class of pyrrolidones, especially N-methyl-2-pyrrolidone, sulfoxides, especially dimethyl sulfoxide (DMSO), formamides, especially dimethyl formamide (DMF), and/or 1-dodecylazacycloheptan-2-one or laurocapran (Azon) and/or derivatives are present in the dosage form.

The dosage form according to the invention is especially preferably characterised in that exclusively dimethylethylene urea is contained as penetration accelerator in the dosage form.

The present invention also relates to a dosage form as described above for use as a medicament.

The present invention further relates to the use of dimethylethylene urea as a penetration accelerator for increasing the skin penetration of active pharmaceutical ingredients having a logP 3, preferably greater than 3.2 or 3.4 or 3.6 or 3.8 or 4 or 4.2 or 4.4 or 4.6 or 4.8 or 5 or 6 or 7.

### DESCRIPTION OF THE DRAWINGS

FIG. 1: Comparison of in vitro permeation profiles of progesterone in various transdermal enhancer systems.

FIG. 2: Comparison of in vitro permeation profiles of felodipine in various transdermal enhancer systems.

FIG. 3: Comparison of in vitro permeation profiles of curcumin in various transdermal enhancer systems.

FIG. 4: Comparison of in vitro permeation profiles of olanzapine in various transdermal enhancer systems.

FIG. 5: TTS as a single-layer matrix system with a self-adhesive polymer matrix based on acrylate type Durotak™ 2054 (Henkel, Düsseldorf) and the non-adhesive polymer type Eudragit™ E100 (Röhm, Darmstadt), as is preferably used for the administration of olanzapine according to Example 4.
(1) Carrier film, for example made of polyethylene terephthalate
(2) Single-layer adhesive matrix polymer; contains the active ingredient, enhancer, and polymer excipient.
(3) Protective film, for example made of polyethylene; is removed before application.

FIG. 6: Schematic representation of a transdermal application system for saturated active ingredient enhancer solutions with occlusion effect, as preferably used for Examples 1 to 3 for the administration of progesterone, felodipine and curcumin.
(4) Backing, for example PET film coated with silicone bio PSA # 4302 (Dow Corning)
(5) Self-adhesive adhesive ring (=adhesive eye) for example made of polyethylene foam and synthetic rubber as adhesive, for fixing the backing (for example Duplocoll® 5009 from Lohmann, Neuwied, (Germany)).
(6) Textile and needled non-woven fabric, preferably with a weight of up to 150 g/m², with regard to absorption behaviour suitable for the absorption of the active ingredient preparation (solution, gel, ointment) for example made of polyester or viscose, such as Paramoll® N260/150 (Lohmann, Neuwied (Germany)) or TWE non-woven 120 (TWE, Dierdorf (Germany)). The application area is, for example, 1.165 cm².
(7) Self-adhesive polyurethane film as control membrane and for fixing the system to the skin, for example Suprasorb® (Lohmann & Rauscher, Neuwied (Germany)).
(8) Diffusion membrane, here preferably native human skin.

FIG. 7:

Comparison of in vitro permeation profiles of olanzapine in various transdermal enhancer systems.

FIG. 8:

TTS as a single-layer matrix system with a self-adhesive polymer matrix based on a silicone adhesive of the type Bio-PSA™ 7-4301 (Dow Corning Corp., Midland, Mich., USA), as preferably used for the administration of olanzapine according to Example 5.
(1) Carrier film, for example made of polyethylene terephthalate.
(2) Single-layer adhesive matrix polymer; contains the active ingredient, enhancer, and polymer excipient.
(3) Protective film, for example made of polyethylene; is removed before application.

The invention will be described in greater detail hereinafter on the basis of non-limiting examples.

### EXAMPLES

The following test series with the various penetration accelerators and for the selected active ingredients were carried out in the context of a typical in vitro permeation using Franz diffusion cells. The used acceptor medium was replaced completely for a new one at predetermined replacement times, and the content of permeated active ingredient amount in these acceptor solutions was determined by means of HPLC. As a comparison, the best penetration accelerators from each test series were used, applied as saturated active ingredient penetration accelerator solutions in Examples 1 to 3 and in Example 4 as a transdermal therapeutic system.

### Example 1

Active ingredient: Progesterone (logP 3.87)

Skin model: Human skin; dermatomised at 500 μm, (female abdomen, date of birth 1968).

Acceptor: Phosphate buffer pH 5.5+0.1% NaN₃+3% gamma-cyclodextrin as solubiliser, which is needed because progesterone is very lipophilic and therefore almost insoluble in water.

Loading: saturated solution of progesterone in DMEU, donor volume 150 μl as direct application to the epidermal skin surface (corresponds to a loading concentration of c=27.7 mg/cm², which is quite high compared to other penetration accelerators, for example only 6.4 mg/cm² for dimethyl isosorbide and 2.1 mg/cm² for dipropylene glycol).

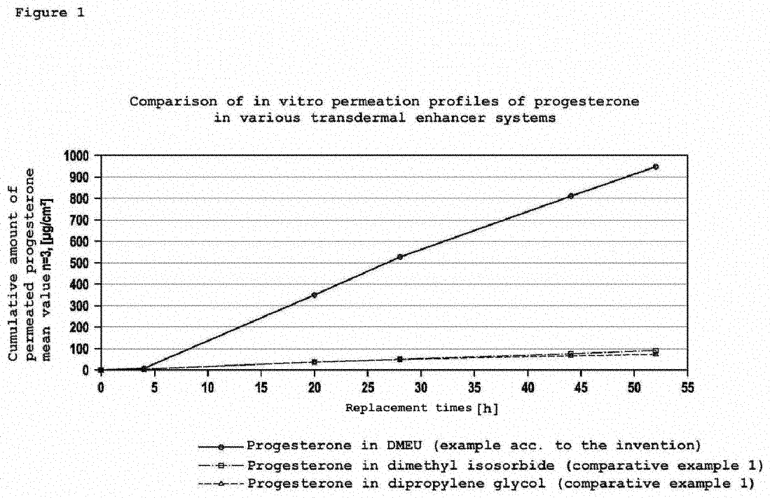

The cumulative amount of permeated progesterone at the predetermined exchange times is shown in FIG. 1.

The penetration acceleration of DMEU is clearly superior to that of the comparative compounds. In relation to the 52 h value or the flux rate in steady state, the effect of DMEU is greater by a factor of about 10 (dimethyl isosorbide) or 13 (dipropylene glycol).

### Example 2

Active ingredient: Felodipine (logP 3.86)

Skin model: Human skin; dermatomised at 500 μm, (female abdomen, date of birth 1985).

Acceptor: Phosphate buffer pH 5.5+0.1% NaN₃+2 wt. % Tween® 20 as solubiliser, which is needed because felodipine is very lipophilic and therefore almost insoluble in water.

Loading: Saturated solution of felodipine in DMEU, donor volume 150 μl as direct application to the epidermal skin surface (corresponds to a loading concentration of c=6.0 mg/cm², which is quite high compared to others).

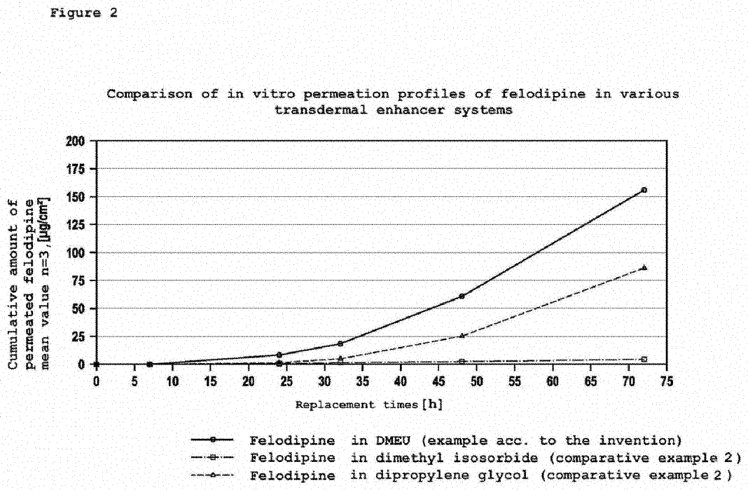

The cumulative amount of permeated felodipine at the predetermined replacement times is shown in FIG. 2.

The penetration acceleration of DMEU is clearly superior to that of the comparative compounds. In relation to the 24h value or the flux rate in steady state, the effect of DMEU is greater by a factor of about 10 (dimethyl isosorbide) or 4 (dipropylene glycol).

### Example 3

Active ingredient: Curcumin (test active ingredient; logP 3.62)

Skin model: Human skin; dermatomised at 500 μm, (female abdomen, date of birth 1979).

Acceptor: Phosphate buffer pH 5.5+0.1% NaN₃+2 wt. % Tween® 20 as solubiliser, which is needed because curcumin is very lipophilic and therefore almost insoluble in water.

Loading: Saturated solution of curcumin in DMEU, donor volume 150 μl as direct application to the epidermal skin surface (corresponds to a loading concentration of 12.75 mg/cm², which is very high compared to that of just 1.29 mg/cm² for dimethyl isosorbide).

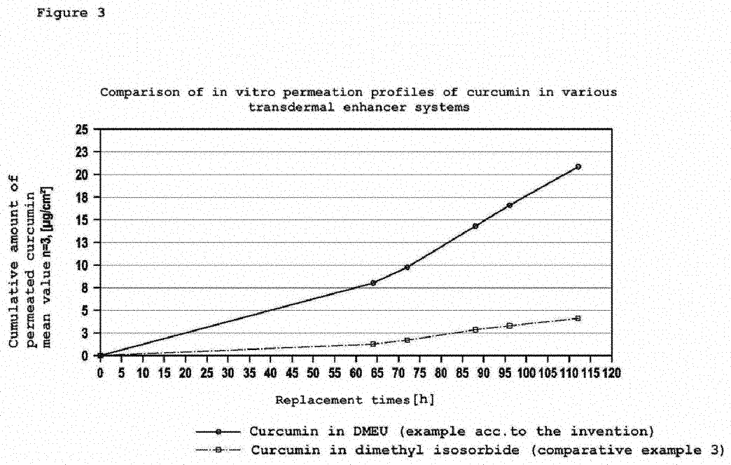

The cumulative amount of permeated curcumin at the predetermined replacement times is shown in FIG. 3.

The penetration acceleration of DMEU is clearly superior to that of the comparative compound. In relation to the 112h value or the flux rate in steady state, the effect of DMEU is greater than the effect of dimethyl isosorbide by a factor of about 5.

### Example 4

Active ingredient: Olanzapine (logP 4.1)

Skin model: Human skin; non-dermatomised, full skin (female abdomen, date of birth 1967).

Acceptor: Phosphate buffer pH 5.5+0.1% NaN₃+2 wt. % Tween® 20 as solubiliser, which is needed because olanzapine is very lipophilic and therefore almost insoluble in water.

Loading: Active ingredient loading 10 wt. % in the transdermal therapeutic system, corresponding to 6 mg/cm² (At a wet-extraction line thickness of 3000 μm). This is very high compared to the transdermal therapeutic system with Eutanol G as penetration accelerator with only 0.26 mg/cm² loading.

Loading system: Transdermal therapeutic system as a single-layer matrix system with a self-adhesive polymer matrix based on the acrylate type Durotak™ 2054 (Henkel, Düsseldorf) and the non-adhesive polymer type Eudragit™ E100 (Röhm, Darmstadt) in a ratio of 4:1 as excipient with 18 wt. % DMEU as penetration accelerator.

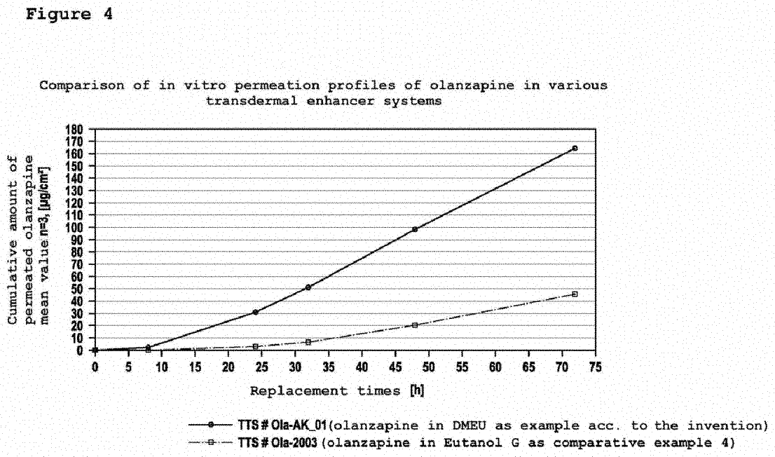

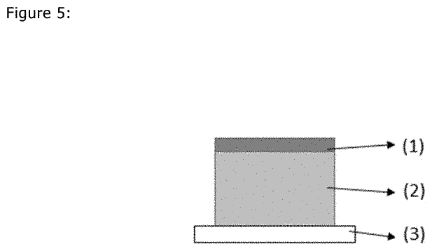

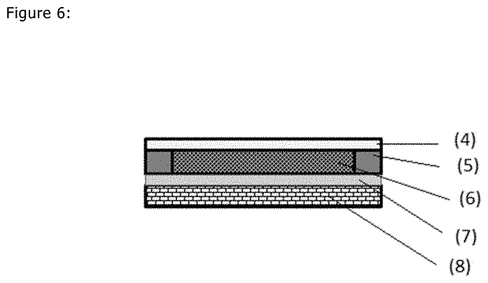

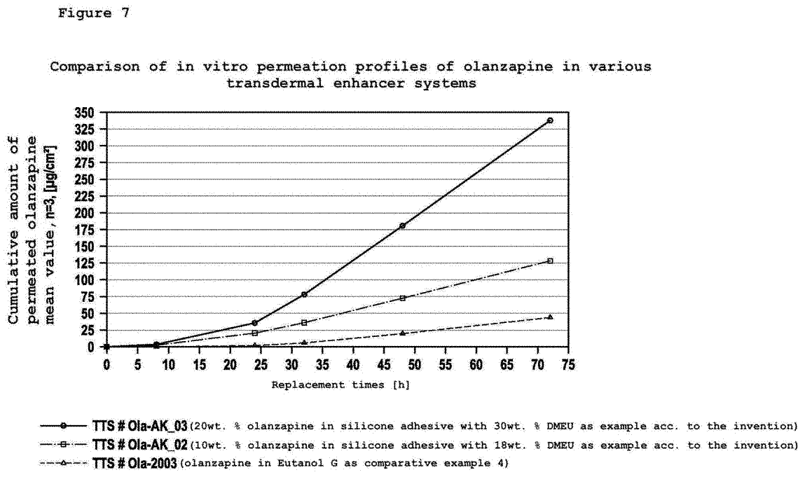

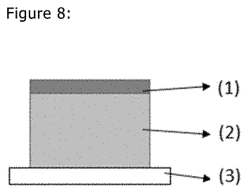

The cumulative amount of permeated curcumin at the predetermined replacement times is shown in FIG. 4.

The penetration acceleration of DMEU is clearly superior to that of the comparative compound. In relation to the 72 h value or the flux rate in steady state, the effect of DMEU is greater than the effect of Eutanol G by a factor of about 3.6.

### Example 5

Active ingredient: Olanzapine (logP 4.1)

Skin model: Human skin; non-dermatomised, full skin (female abdomen, date of birth 1967).

Acceptor: Phosphate buffer pH 5.5+0.1% NaN₃+2 wt. % Tween® 20 as solubiliser, which is needed because olanzapine is very lipophilic and therefore almost insoluble in water.

Loading: Active ingredient loading 10 wt. % in the transdermal therapeutic system, corresponding to 6.47 mg/cm² (At a wet-extraction line thickness of 3000 μm). This is very high compared to the transdermal therapeutic system with Eutanol G as penetration accelerator with only 0.26 mg/cm² loading.

Loading system: Transdermal therapeutic system as a single-layer matrix system with a self-adhesive polymer matrix based on silicone type BIO-PSA™ 7-4301 (Dow Corning Corp., Midland, Mich., USA) with 18 wt. % DMEU as penetration accelerator (see FIG. 8).

The cumulative amount of permeated olanzapine at the predetermined replacement times is shown in FIG. 7.

The penetration acceleration of DMEU is clearly superior to that of the comparative compound. In relation to the 72 h value or the flux rate in steady state, the effect of DMEU is greater than the effect of Eutanol G by a factor of about 2.8.

### Example 6

Active ingredient: Olanzapine (logP 4.1)

Skin model: Human skin; non-dermatomised, full skin (female abdomen, date of birth 1985).

Acceptor: Phosphate buffer pH 5.5+0.1% NaN₃+2 wt. % Tween® 20 as solubiliser, which is needed because olanzapine is very lipophilic and therefore almost insoluble in water.

Loading: Active ingredient loading 20 wt. % in the transdermal therapeutic system, corresponding to 8.5 mg/cm² at a wet extraction line thickness of 1200 μm. This loading is very high compared to the transdermal therapeutic system with Eutanol G as penetration accelerator with only 0.26 mg/cm² loading.

Loading system: Transdermal therapeutic system as a single-layer matrix system with a self-adhesive polymer matrix based on silicone type BIO-PSA™ 7-4301 (Dow Corning Corp., Midland, Mich., USA) with 30 wt. % DMEU as penetration accelerator (see FIG. 8).

The cumulative amount of permeated olanzapine at the predetermined replacement times is shown in FIG. 7.

The penetration acceleration of DMEU is clearly superior to that of the comparative compound. In relation to the 72 h value or the flux rate in steady state, the effect of DMEU is greater than the effect of Eutanol G by a factor of about 6.

1. A dosage form for transdermal administration of at least one active pharmaceutical ingredient, comprising at least one active pharmaceutical ingredient with a logP≥3 and at least one penetration accelerator, characterised in that the at least one penetration accelerator comprises dimethylethylene urea.

2. The dosage form according to claim 1, characterised in that the dosage form comprises a transdermal therapeutic system, a gel, a lotion, an ointment and/or a cream.

3. The dosage form according to claim 1, characterised in that the at least one active pharmaceutical ingredient with a logP≥3 has a water solubility of less than 0.01 mg/ml (at 20° C.).

4. The dosage form according to claim 1, characterised in that the at least one active pharmaceutical ingredient is selected from the group consisting of hypnotics, sedatives, antiepileptics, analeptics, psychoneurotropic drugs, neuroleptics, neuro muscle blockers, antispasmodics, antihistamines, antiallergics, cardiotonics, antiarrhythmics, diuretics, hypotensives, vasopressors, antitussives, expectorants, analgesics, thyroid, hormones, sexual hormones, glucocorticoid hormones, antidiabetics, antitumour drugs, antibiotics, chemotherapeutics, narcotics, anti Parkinson drugs, anti Alzheimer drugs and/or triptans.

5. The dosage form according to claim 1, characterised in that the dosage form represents a transdermal therapeutic system, characterised in that the transdermal therapeutic system has a backing and a matrix layer containing the at least one active pharmaceutical ingredient with a logP≥3.

6. The dosage form according to claim 5, characterised in that the at least one active penetration accelerator dimethylethylene urea is provided in the matrix layer in an amount of from 10 to 30 wt. % in relation to the active ingredient containing matrix layer.

7. The dosage form according to claim 5, characterised in that the matrix layer comprises at least one polymer selected from the group consisting of polyacrylates and/or polymethacrylates, natural and/or synthetic rubbers, polysiloxanes, styrene butadiene block copolymers, isobutylene and/or ethylene vinyl acetate copolymers.

8. The dosage form according to claim 5, characterised in that the dosage form represents a transdermal therapeutic system which is formed as membrane systems, wherein the at least one active pharmaceutical ingredient is present in the matrix layer in a reservoir, from which the at least one active pharmaceutical ingredient can be dispensed through a porous control membrane covering the reservoir.

9. The dosage form according to claim 8, characterised in that the control membrane comprises a polymer film, wherein the polymer forming the basis of the polymer film is selected from polyethylene, polypropylene, polyurethane, silicone and/or copolymers of ethylene and vinyl acetate.

10. The dosage form according to claim 5, characterised in that the matrix layer comprises further excipients selected from the group consisting of plasticisers, crystallisation inhibitors, stabilisers, antioxidants and/or neutralisers.

11. The dosage form according to claim 5, characterised in that the at least one active pharmaceutical ingredient is present in the matrix layer in an amount of from 0.1 to 50 wt. %, in relation to the weight of the matrix layer.

12. The dosage form according to claim 5, characterised in that the transdermal therapeutic system has a loading with the at least one active pharmaceutical ingredient of greater than 6 mg/cm2.

13. The dosage form according to claim 5, characterised in that exclusively dimethylethylene urea is contained as penetration accelerator in the dosage form.

14. The dosage form according to claim 1, for use as a medicament.

15. A method for the administration of at least one active pharmaceutical ingredient with a logP≥3 in combination with at least one penetration accelerator, wherein the penetration accelerator comprises dimethylethylene urea.

16. The dosage form according to claim 5, characterised in that the at least one active penetration accelerator dimethylethylene urea is provided in the matrix layer in an amount of from 12 to 25 wt. in relation to the active-ingredient-containing matrix layer.

17. The dosage form according to claim 5, characterised in that the at least one active penetration accelerator dimethylethylene urea is provided in the matrix layer in an amount of from 15 to 18 wt. % in relation to the active-ingredient-containing matrix layer.
